(19) United States
(12) Patent Application Publication
(10) Pub. No.: US 2008/0171928 A1
(43) Pub. Date: Jul. 17, 2008

(54) METHODS AND SYSTEMS OF CLASSIFYING BLEEDING EVENTS

(76) Inventors: Victor L. Serebruany, West Friendship, MD (US); Daniel Atar, Oslo (NO)

Correspondence Address: ANTOINETTE G. GIUGLIANO, P.C., 100 Cummings Center, Suite 342G, Beverly, MA, 01915, US

(21) Appl. No.: 11/623,080

(22) Filed: Jan. 14, 2007

(51) Int. Cl. A61B 5/00

(52) U.S. Cl. 600/371

(57) ABSTRACT

The present invention includes methods and systems for classifying and assigning a measurement to a bleeding event exhibited by an individual. The system includes two or more categories, wherein each category has a different level of severity; two or more bleeding events assigned to each category; and a set of points for each category. Points are assigned to a category depending on the severity of the category, wherein the more severe categories have a greater number of points assigned to it. The classification includes a categorical designation that indicates the category from which the points were assigned.

Accordingly, the methods for assigning a measurement to at least one bleeding event exhibited by an individual include assigning one or more points to the bleeding event according to a classification system, and optionally assigning a categorical designation to the measurement.

### BACKGROUND OF THE INVENTION

Present classifications of bleeding events used in antithrombotic, anticoagulant, and/or antiplatelet clinical trials and scenarios are often based on criteria developed by the Thrombolysis in Myocardial Infarction (TIMI) study group or the Global Use of Strategies To Open coronary arteries (GUSTO) investigators. Such classifications are used to better categorize hemorrhagic events after therapy with certain thrombolytic agents. However, these classification systems do not wholly account for or accurately reflect certain bleeding complications, in particular, those resulting from interventional procedures (e.g., stents) and those complications resulting from the administration of certain antithrombotic, anticoagulant, and/or antiplatelet drugs.

Hence, a need exists for better, more comprehensive methods for classifying bleeding events. In particular, a need exists for methods and systems for assigning a measurement and standardized registration to bleeding events relating to intervention procedures and administration of antithrombotic and antiplatelet therapies.

### SUMMARY OF THE INVENTION

The present invention encompasses methods for assigning a measurement to at least one bleeding event exhibited by an individual. The method includes assigning one or more points to the bleeding event according to a classification system, wherein the points are determined by a category to which the bleeding event belongs. The classification system has at least one category, each category comprises at least two bleeding events. The method further includes assigning a second designation, referred to as a “categorical designation,” to the measurement. The classification designation is an indication of the category to which the bleeding event belongs. In one aspect, the assignment of one or more points is repeated for each bleeding event of the individual. In another embodiment, the methods include summing (e.g., obtaining a total of) the points for all bleeding events exhibited by the individual over a period of time to thereby obtain a total measurement.

In particular, the present invention includes methods for assigning a measurement to at least one bleeding event exhibited by an individual in accordance with a classification system. The system includes at least two categories, wherein each category signifies a level of severity of the bleeding event; a set of points that are determined by a category to which the bleeding event belongs; and at least two bleeding events belonging to each category. The methods involve comparing the bleeding event exhibited by the individual with bleeding events for each category; and assigning points of the category to which the bleeding event belongs to the bleeding event of the individual. If the bleeding event is not present in the category, then 0 points are assigned. In an embodiment, three categories can be used (e.g., a mild category, a moderate category, and a severe category; or a superficial category, an internal category and an alarming category). The bleeding event included in the categories include, e.g., easy bruising, bleeding from small cuts, petechia, ecchymosis, hematoma, epistaxis, blood loss from mouth, vagina, melena, eye bleed, hematuria, hematemesis, hemoptysis, transfusion needed, intracranial, life threatening. In a particular embodiment, a first category includes easy bruising, bleeding from small cuts, petechia, ecchymosis; a second category has hematoma, epistaxis, blood loss from mouth, vagina, melena, eye bleed, hematuria, hemoptysis, hematemesis; and a third category includes transfusion needed, intracranial, life threatening.

In particular, the present invention includes methods for assigning a measurement to at least one bleeding event exhibited by an individual by assigning one or more points to the bleeding event according to a classification system, wherein the points are determined by a category to which the bleeding event belongs. The assignment of a measurement is done according to the following the classification system shown in Table 1:

TABLE 1

| Severity | Criteria | Points |
|---|---|---|
| Superficial | Easy bruising, Bleeding from small cuts, Petechia, | 1 |
| | Ecchymosis | |
| Internal | Hematoma, Epistaxis, Blood loss from Mouth, | 3 |
| | Vagina, Melena, Eye Bleed, Hematuria, | |
| | Hemoptysis, hematemesis | |
| Alarming | Transfusion Needed, Intracranial, Life Threatening | 6 |

Zero “0” points are assigned when the bleeding event of the individual does not belong to the category.

Using the steps described herein, the present invention includes methods of treating an individual who is undergoing or underwent a medical procedure or treatment, or individuals at risk for a bleeding event. This is accomplished by, at one or more time points, assigning points to a bleeding event of the individual, as described herein, and comparing the measurements. An increase in the measurements indicates an increase in bleeding; and a decrease in the measurements indicates a decrease in bleeding.

The methods of the present invention can also be employed in conducting a trial or study having at least two individuals who are undergoing examination or scrutinization in a specified period of time, and who may experience one or more bleeding events, or who are at risk for one or more bleeding events. The methods include assigning points to a bleeding event of each individual, as described herein, and to thereby obtain a measurement; and comparing said measurements. The measurement indicates the extent of bleeding for the individual. Comparing the measurements can involve comparing measurements of individuals within a group, comparing measurements of one group with measurements of one or more other groups of the trial, or both.

The present invention further includes systems of classifying one or more bleeding events in an individual. The system embodies two or more categories, wherein each category has a different level of severity; two or more bleeding events assigned to each category; and a set of points for each category. A greater number of points are assigned to a category having a greater level of severity, and a lesser number of points is assigned to a category having a lesser level of severity. The system further includes a categorical designation that indicates the category from which the points were assigned. In another embodiment, the system of the present invention has categories, bleeding events and a set of points as shown in Table 1 (above). This system includes a categorical designation that comprises “S” for Superficial, “I” for Internal and “A” for Alarming.

The present invention has several advantages. The methods and classification systems of the present invention allow medical professionals to assign a numerical value to a patient's bleeding state. This allows the medical professional to clearly and more easily convey the extent of bleeding in an individual, to easily compare datasets generated under this method, and to triage the severity of the bleeding event to tailor appropriate therapy. Additionally, since it is a measurement, statistical analysis can be performed, and compared. This makes the methods and system of the present invention well suited for studies and trials e.g., for new drug therapy or new coronary procedures.

### DETAILED DESCRIPTION OF THE INVENTION

The present invention relates to methods and systems of classifying bleeding events to convey the extent of bleeding in an individual. The classification system has at least one category (e.g., two or more categories) of severity and each category has at least two bleeding events assigned to it. The categories each have a number of points assigned to it as well, and the number of points correlates to the severity of the category. More severe categories have a greater amount of points, and less severe categories have a fewer amount of points, as further described herein. A bleeding event of an individual is compared to the bleeding events for each category, and is assigned the number points of the category to which the bleeding event belongs, to obtain a measurement (referred to herein as a “BleedScore™” measurement). The measurements obtained for each bleeding event for an individual can be further classified, as described herein, or summed to obtain an overall total score. The individual and/or total BleedScore™ conveys the extent of the bleeding occurring in an individual. The term “BleedScore™” refers to the classification, amounts, points, and/or designations, obtained using the methods and/or system of the present invention.

Accordingly, the steps of the methods of the present invention involve assigning points to a bleeding event occurring or that occurred in an individual. The method includes determining a category to which the bleeding event belongs. The category, in an embodiment, relates to levels of the severity or magnitude of the bleeding event. Any name for such categories can be used so long as the severity of the category is conveyed. Severity of the category relates to the degree of bleeding that occurs for the bleeding events for that category, as further described herein. As used in the classification system described in the Exemplification, three categories were created and named as Superficial, Internal, and Alarming. Another example of a naming convention that can be used is Mild, Moderate, and Severe. The names of the categories include full names, abbreviations, acronyms, and any other numeral, symbolic, or letter designations. The number of categories ranges from about 2 categories to about 10 categories (e.g., about 3, 4, 5, 6, 7, 8, or 9), and in one embodiment, about 3 categories are used.

Assigned to each category is at least one bleeding event. A bleeding event is any event in which a leaking of a blood vessel occurs, and includes, but is not limited to, bleeding complications that arise as the result of treatments, drug therapy or procedures (e.g., coronary procedures). In an embodiment, the methods and systems relate to bleeding complications that result from interventional techniques (e.g., stents) and antithrombotic and antiplatelet therapies (e.g., extended release dipyridamol, warfarin, heparin, hirudin, clopidogrel, platelet GPIIb/IIIa inhibitors, low molecular weight heparins, or bivalirudin). Examples of bleeding events include Easy Bruising (e.g., where more bruising occurs than expected as compared to an individual not at risk for bleeding); Bleeding from Small Cuts (e.g., bleeding that is more than expected as compared to an individual not at risk for bleeding); Petechia; Ecchymosis; Hematoma; Epistaxis; Blood loss from Mouth, Vagina, Melena; Eye Bleed; Hematuria; Hemoptysis, hematemesis; events needing a transfusion (e.g., Transfusion Needed); Intracranial bleeding; Life Threatening bleeding (e.g., bleeding that if left untreated can cause death). Other bleeding events can be used in the classification methods and systems of the present invention, including those now know, or those developed, discovered or characterized as such in the future.

In the example above in which 3 categories, Superficial, Internal and Alarming were used, the bleeding events can be assigned to these categories as follows:
Superficial—Easy bruising, Bleeding from small cuts, Petechia, Ecchymosis
Internal—Hematoma, Epistaxis, Blood loss from Mouth, Vagina, Melena, Eye Bleed, Hematuria, Hemoptysis, hematemesis
Alarming—Transfusion Needed, Intracranial, Life Threatening.

The methods and systems of the present invention further include assigning points to the bleeding event depending on the category to which the bleeding event is assigned. The degree of the severity relates to the number of points assigned to that category. Severity relates to the degree of which a bleeding event threatens the health of the individual undergoing the event. Severity can be established by a professional trained in that area and include, physicians, researchers, nurses, and the like. The greater the degree of the severity, the more points are assigned to the category. Categories that are less severe have less points assigned to it, as compared to categories having bleeding events that are more severe. Accordingly, bleeding events are preferably assigned to categories according to their clinical importance or outcome. Bleeding events that generally result in severe complications or severely compromise an individual's health are assigned to a category having more points, whereas bleeding events that are considered to be superficial or mild are assigned to a category have less points. Similarly, an intermediate category is assigned a number of points in between these two amounts, and contains bleeding events that are considered internal bleeding, or of a moderate severity.

Any number of points can be assigned to each category as long as the number of points assigned correlates to the severity of the category. The number of points ranges from 0 to 1000. In a particular embodiment, the bleeding event of the individual is compared to the bleeding events contained in each category. If the bleeding event is present in the category, then the bleeding event is assigned the point or points for that category. If the bleeding event is not present, then in an embodiment, the bleeding event is assigned 0 points for that category. In the above example, the Superficial category is assigned 1 point, the Internal category is assigned 3 points, the Alarming category is assigned 6 points, and 0 is assigned if the bleeding event is not present in that category.

A person utilizing the classification system and methods of the present invention assigns a number points to the bleeding event, depending on the category to which it belongs. In the above example, bleeding events of an individual consisting of easy bruising, eye bleeding, blood loss from the mouth, and intracranial bleeding would be assigned a 1, 3, 3, and 6, respectively.

Additionally, the number of points assigned to a single bleeding event can be added up to get a total score for the individual. Hence, the total BleedScore™ amount given to the individual in the example would be 13. Based on this example, a score of 13 indicates a large amount of internal bleeding in an individual. This classification system allows for the possibility of several events (e.g., two or more) in a less severe category (e.g., in a Superficial or Internal category) to add up to indicate significant overall bleeding in the individual.

The classification system and methods of the present invention further include assigning a second, category designation, to the bleeding event in addition to the points. The second designation relates to the category from which the bleeding event belongs. The designation can be a letter, symbol, or any other designation that indicates the category. This designation is a second indicator of the severity of the bleeding event. The BleedScore™ points are added for that specific category and then assigned the second, category designation. In the above example, the first letter of the category name was used in a subscript format to indicate the category from which the points came. Specifically, the bleed category can be expressed, in the above example, as follows: “S” for Superficial, “I” for Internal, and “A” for Alarming. Hence, the BleedScore™ classification in the example above is 1_{S}, 6_{I}, 6_{A}. This second, categorical designation is of the bleed event is optional, and, in an embodiment, the classification system of the present invention can utilize the numerical scoring methods alone.

The system of the present invention includes about 2 or more categories of bleeding events, about 2 or more bleeding events assigned to each category, a set of points wherein a number of points are assigned to each category according to its severity, and optionally, a second, categorical designation (e.g., that is not a number) to signify from which category the points were assigned, as described herein.

The methods and system of the present invention can be used to assess the efficacy of a treatment and/or a procedure, or to treat an individual. In such a case, a BleedScore™ measurement can be taken at one or more time points such as before, during and/or after the treatment/procedure. More than one measurement can be taken at any combination of these time points. An increase in the BleedScore™ measurement indicates that an increase in bleeding is occurring, wherein a decrease indicates that a decrease in bleeding is occurring. An increase in bleeding indicates that the treatment or procedure is not working, while a decrease indicates that it is working.

Additionally, the methods of the present invention include assessing a patient who has undergone one or more bleeding events which are independent of a specific treatment or procedure by utilizing one or more of the steps and/or the system described herein. The methods and systems of the present invention can be used on individual that are at risk for a bleeding event. At risk for a bleeding event refers to an individual who has a medical status or history that indicates bleeding may occur or is likely to occur.

The methods of the present invention further encompass applying the methods and systems for assigning a BleedScore™ measurement to patients that are part of a trial or study, e.g., relating to procedures and/or therapies in which bleeding is a concern or side effect. Accordingly, a BleedScore™ assessment can be performed on each patient in the trial thereby providing a standardized way to compare each patient to one another, and each group of patients to other groups of patients in a given, specified period of time. Total scores can be taken, and statistical analysis including averages, medians, statistical significance can be calculated.

A description of preferred embodiments of the invention follows.

### Exemplification

### Summary:

Present classifications of bleeding events used in antithrombotic, anticoagulant and/or antiplatelet clinical trials are based on the criteria developed by the Thrombolysis in Myocardial Infarction (TIMI) study group and the Global Use of Strategies To Open coronary arteries (GUSTO) investigators. Being introduced about decade ago, both classifications used the criteria to categorize hemorrhagic events after therapy with thrombolytic agents. Recent advances in interventional cardiology, resulting in a domination of percutaneous intracoronary procedures over systemic drug-induced thrombolysis, substantially changed the clinical characteristics and magnitude of bleeding complications. Moreover, disturbances of the coagulation cascade, as well as platelet inhibition caused directly by antithrombotic and antiplatelet agents share very specific and well-recognized clinical features not reflected in the existing classifications. In fact, bleeding events after aspirin or clopidogrel, and especially those after more delicate antiplatelet regimens with dipyridamol used in patients after ischemic stroke or TIA, are close to impossible to classify by the present guidelines, other than categorically triaging them altogether to the “minor” category. Uniting entirely different bleeding events as “minor” underrates their importance and diminishes affiliated risks, creating an illusion that they do not require monitoring or/and changes in antiplatelet or antithrombotic regimens. In reality such unrecognized and unreported mild complications can, in certain instances, transform into more serious bleeds, or lead to non-compliance. Unauthorized withdrawal from antiplatelet agents in turn cause rebound platelet activation, and higher risk for secondary vascular events. Hence, introduced herein is a new classification of bleeding events (the “BleedScore”™) based on a point accumulation depending on the severity of hemorrhage, is more suitable for the assessment of modern more delicate antithrombotic and antiplatelet therapies, particularly for their realistic assessment in clinical trials.

BleedScore™ Classification Method and Comparison with Existing Classification Methods:

Antithrombotic, anticoagulant and antiplatelet therapies have been the focus of extensive clinical investigations over the last two decades. A variety of agents and regimens have been advanced for the prevention and treatment of vascular disease. Numerous experimental drugs are currently being tested, and some of them will be approved to expand the available treatment armamentarium. Unquestionably, therapies inhibiting coagulation and primary hemostasis are associated with an increased risk of bleeding. However, the present classifications of bleeding complications are often of limited clinical utility, particularly with regard to categorizing and triaging mild-to-moderate hemorrhagic events.

A common classification used to report bleeding severity is the one developed by the Thrombolysis in Myocardial Infarction (TIMI) study group. Criteria developed by the Global Use of Strategies en coronary arteries (GUSTO) study group are less commonly adopted. These classifications are presented in Table 2 and 3.

TABLE 2
TIMI classifications of bleeding severity

| Classification | Severity | Criteria |
|---|---|---|
| TIMI | Major | Intracranial bleeding. Overt bleeding |
| | | with a decrease in hemoglobin ≧5 g/dL |
| | | or decrease in hematocrit ≧15% |
| | Minor | Spontaneous gross hematuria. |
| | | Spontaneous hematemesis. Observed |
| | | bleeding with decrease in |
| | | hemoglobin ≧3 g/dL but ≦15% |
| | Insignificant | Blood loss insufficient to meet criteria |
| | | listed above |

TABLE 3
GUSTO classifications of bleeding severity

| Classification | Severity | Criteria |
|---|---|---|
| GUSTO | Severe | Deadly bleeding. Intracerebral bleeding or |
| | | substantial hemodynamic compromise |
| | | requiring treatment |
| | Moderate | Bleeding requiring transfusion |
| | Mild | Other bleeding not requiring transfusion |
| | | or causing hemodynamic |
| | | compromise |

The TIMI criteria are more precise and specific, especially when used together with an index referred to as the “Landefeld transfusion” index, while the GUSTO classification tends to be more subjective. These classifications, introduced about a decade ago, were indeed well suited for the thrombolytic era. At that time, stents, extended release dipyridamol, clopidogrel, platelet GPIIb/IIIa inhibitors, low molecular weight heparins, or bivalirudin, were largely unavailable and not widely used.

Certain recent advances in percutaneous interventional techniques, paired with modern pharmacotherapy of atherothrombosis, have substantially changed the clinical presentations of bleeding events. Moreover, alterations of primary hemostasis, as well as platelet inhibition caused directly by antiplatelet agents, exhibit very specific and well-recognized clinical features, which, however, are not reflected in the presently used classifications. In fact, bleeding episodes after clopidogrel, and especially those after more delicate antiplatelet regimens such as dipyridamol used in patients after ischemic stroke and Transient Ischemic Attack (TIA), or aspirin are very difficult if possible to classify by the present guidelines, other than ultimately triaging them altogether to the “insignificant” category. Therefore, placing these different bleeding events “onto one shelf” underrates their importance and diminuates their associated risks, creating an illusion that they do not require monitoring or/and changes in the treatment regimens. In reality such unrecognized and unreported events may transform into more severe bleeding complications, or/and be responsible for substantial non-compliance. Unauthorized withdrawal from antiplatelet agent(s) has been recognized as a major cause of rebound platelet activation, and subsequent higher risk for secondary vascular events. Another confusing issue is the association of certain antithrombotic/anticoagulant/antiplatelet regimens with intracranial hemorrhages. The latter are the cornerstones of both TIMI (major) and GUSTO (severe) classifications. While intracranial bleedings indeed represent the major life-threatening complication of thrombolytic therapy, the use of antiplatelet agents, even the most potent of them (GPIIb/IIIa inhibitors), is not associated with a substantial increase of intracranial bleeding rates.

Taken together, the proper assessment of the safety profile of mild-to-moderate antiplatelet or/and antithrombotic regimens is difficult, if not impossible, using the presently available classifications. Instead, the medical community is lulled by endless and somewhat senseless claims of “low incidence of bleeding events”, even in studies testing aggressive combination regimens. Moreover, present classifications make it difficult to compare the safety of antiplatelet agents in head-to-head comparison trials. Nonetheless, these safety data are important for a realistic appreciation of the bleeding risks inherent to the established agents, and, more so, may be key for an adequate safety risk assessment for new agents. As an exemplification of the problem, the discrepancies in bleeding assessments during dual antiplatelet therapy with clopidogrel and aspirin as reported in recent major clinical trials (Table 4) were compiled.

TABLE 4
Incidence of bleeding events during dual antiplatelet therapy
in patients after vascular events.*

| Bleeding Type | Reported Incidence (%) (TRIAL NAME) |
|---|---|
| TIMI Major | 3.7 (CURE)**; 4.8 (CREDO); 2.0 (MATCH)**; 1.9 |
| | (CLARITY); 0.58 (COMMIT); 0.8 (JUMBO); |
| TIMI Minor | 5.1 (CURE); 1.7 (CREDO); 3.0 (MATCH); |
| | 1.6 (CLARITY); |
| TIMI | None reported |
| Insignificant | |
| GUSTO Severe | 2.2 (CURE); 1.7 (CHARISMA) |
| GUSTO | 1.2 (CURE); 2.1 (CHARISMA) |
| Moderate | |
| GUSTO Mild | None reported |
| Total*** | 8.5 (CURE)**; 3.5 (JUMBO) |

*all patients received clopidogrel loading 300 mg, or/and 75 mg/daily maintenance dose in combination with 75–325 mg/daily aspirin;
**by CURE bleeding definition;
***some patients had more than one bleeding episode

It is unreasonable to expect identical bleeding rates across all trials due to different patient populations, underlying diseases, treatment duration, or use of concomitant medications. However, not only the differences within the same bleeding types are substantial, major trials actually failed to report any bleeding complications in the “Insignificant” (TIMI), or “Mild” (GUSTO) categories. There is not the slightest doubt that the bleeding risk in these categories is in fact the highest, e.g., when a bleeding adverse event such as “easy bruising” was observed in as much as 48% of patients with stable coronary artery disease treated with clopidogrel together with aspirin in one of the recent trials.

A new classification was generated which will allow a better triaging of bleeding disorders, especially at the “low end”, and which at the same time will cover the entire spectrum of bleeding complications. The summary of such classification, referred to herein as “BleedScore™” is presented in Table 5. An important feature is a cumulative or incremental expression of events by adding points up to a resulting score. The accrued points are on an open-ended scale, meaning that there is no upper limit to the point scale. However, a particular index is added to the point-score, expressing the bleeding category from which points were accrued, i.e., “S” for superficial, “I” for internal, and “A” for alarming. In case of more than one bleeding category involved, the triple-index still reflects all three categories applicable for any given patient.

TABLE 5
BleedScore ™ classification

| Severity | Criteria | Points |
|---|---|---|
| Superficial | Easy bruising, Bleeding from small cuts, Petechia, | 1 |
| | Ecchymosis | |
| Internal | Hematoma, Epistaxis, Blood loss from Mouth, | 3 |
| | Vagina, Melena, Eye Bleed, Hematuria, | |
| | Hemoptysis, hematemesis | |
| Alarming | Transfusion Needed, Intracranial, Life Threatening | 6 |

The minimal BleedScore point is 0, each event is added. Points are accrued on an open-ended scale. Bleeding complications are monitored continuously throughout a given trial. For reporting purposes, the 30 contiguous days prior to a given patient assessment (e.g., during a trial study-visit) constitute the index-month for reporting the BleedScore results. A subscript-index (“S”, “I” or “A”) is given at the end of the score, reflecting each category from which points were accrued.

Example of scores: 1_{S}, 0_{I}, 0_{A};
2_{S}, 3_{I}, 6_{A};

For simplified statistical assessment, e.g. in large-scale trials, points can also be combined into a pooled total BleedScore (e.g.; in the examples above: pooled BleedScore 1+0+0=1; 2+3+6=11).

In conclusion, considering the shortcomings of the existing classifications, this approach is will be particularly useful in the determination and meticulous reporting of mild-to-moderate bleeding events associated with modern antiplatelet and antithrombotic therapies, while at the same time accounting for the most severe bleeding complications, too. As a particular recommendation, the BleedScore™ Classification System should be deployed in randomized clinical trials, prospective registries, and post-marketing surveys.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for assigning a measurement of at least one bleeding event exhibited by an individual, wherein the method comprises:
assigning one or more points to the bleeding event according to a classification system, wherein the points are determined by a category to which the bleeding event belongs;
wherein the classification system has at least one category, and each category comprises at least two bleeding events, wherein said points range between about 0 and about 1000 points.

2. The method of claim 1, the method further includes assigning a categorical designation to the measurement, wherein the classification designation is an indication of the category to which the bleeding event belongs.

3. The method of claim 1, wherein the assignment of one or more points is repeated for each bleeding event of the individual

4. The method of claim 3, further including summing the points for all bleeding events exhibited by the individual to thereby obtain a total measurement.

5. A method for assigning a measurement to at least one bleeding event exhibited by an individual according to a classification system that comprises at least two categories, wherein each category signifies a level of severity of the bleeding event; a set of points that are determined by a category to which the bleeding event belongs; and at least two bleeding events belonging to each category; the method comprises:
a. comparing the bleeding event exhibited by the individual with bleeding events for the category; and
b. assigning one or more points to the bleeding event of the individual, wherein the points are assigned based on the category to which the bleeding event belongs; or 0 points are assigned if the bleeding event is not present in the category.

6. The method of claim 5, wherein the categories comprise a mild category, a moderate category, and a severe category.

7. The method of claim 6, wherein the categories comprise a superficial category, an internal category and an alarming category.

8. The method of claim 7, wherein the bleeding event is selected from the group consisting of easy bruising, bleeding from small cuts, petechia, ecchymosis, hematoma, epistaxis, blood loss from mouth, vagina, melena, eye bleed, hematuria, hemoptysis, hematemesis, transfusion needed, intracranial, life threatening.

9. A method for assigning a measurement to at least one bleeding event exhibited by an individual, wherein the method comprises:
assigning one or more points to the bleeding event according to a classification system, wherein the points are determined by a category to which the bleeding event belongs;
wherein the classification system has about three categories, wherein the first category comprises easy bruising, bleeding from small cuts, petechia, ecchymosis; the second category comprises hematoma, epistaxis, blood loss from mouth, vagina, melena, eye bleed, hematuria, hemoptysis, hematemesis; and the third category comprises transfusion needed, intracranial, life threatening.

10. The method of claim 9, the method further includes assigning a categorical designation to the measurement, wherein the classification designation is an indication of the category to which the bleeding event belongs.

11. The method of claim 10, wherein the assignment of one or more points is repeated for each bleeding event in the individual.

12. The method of claim 11, further including summing the points for all bleeding events exhibited by the individual to thereby obtain a total measurement.

13. A method for assigning a measurement to at least one bleeding event exhibited by an individual, wherein the method comprises:
assigning one or more points to the bleeding event according to a classification system, wherein the points are determined by a category to which the bleeding event belongs;
wherein the classification system comprises:

| Severity | Criteria | Points |
|---|---|---|
| Superficial | Easy bruising, Bleeding from small cuts, Petechia, | 1 |
| | Ecchymosis | |
| Internal | Hematoma, Epistaxis, Blood loss from Mouth, | 3 |
| | Vagina, Melena, Eye Bleed, Hematuria, | |
| | Hemoptysis, hematemesis | |
| Alarming | Transfusion Needed, Intracranial, Life Threatening | 6 |

14. The method of claim 13, further including assigning 0 points when the bleeding event of the individual does not belong to the category.

15. A method of treating an individual who is undergoing or underwent a medical procedure or treatment, or who is at risk for a bleeding event; the method comprises:
a. at one or more time points, assigning points to a bleeding event of the individual in accordance with method of claim 1 to thereby obtain a measurement;
b. comparing said measurements;
wherein an increase in the measurements indicates an increase in bleeding; and a decrease in the measurements indicates a decrease in bleeding.

16. A method of conducting a trial having at least two individuals who are undergoing one or more bleeding events, or who are at risk for one or more bleeding events; the method comprises:
a. assigning points to a bleeding event of each individual in accordance with method of claim 1 to thereby obtain a measurement; and
b. comparing said measurements;
wherein the measurement indicates the extent of bleeding for the individual.

17. The method of claim 16, wherein comparing said measurements involves comparing said measurements of individuals within a group, comparing said measurements of one group with measurements of one or more other groups of the trial, or both.

18. A system of classifying one or more bleeding events of an individual, the system comprises:
a. two or more categories, wherein each category has a different level of severity;
b. two or more bleeding events assigned to each category;
c. a set of points for each category, wherein a greater number of points is assigned to a category having a greater level of severity, and a lesser number of points is assigned to a category having a lesser level of severity.

19. The system of claim 18, further including a categorical designation that indicates the category from which the points were assigned.

20. A system of classifying one or more bleeding events of an individual, the system comprises:
a. about three categories, wherein each category has a different level of severity;
b. about three or more bleeding events assigned to each category;
c. a set of points for each category, wherein a greater number of points is assigned to a category having a greater level of severity, and a lesser number of points is assigned to a category having a lesser level of severity
wherein the classification system comprises:

| Severity | Criteria | Points |
|---|---|---|
| Superficial | Easy bruising, Bleeding from small cuts, Petechia, | 1 |
| | Ecchymosis | |
| Internal | Hematoma, Epistaxis, Blood loss from Mouth, | 3 |
| | Vagina, Melena, Eye Bleed, Hematuria, | |
| | Hemoptysis, hematemesis | |
| Alarming | Transfusion Needed, Intracranial, Life Threatening | 6 |

21. The system of claim 20, further including a categorical designation that comprises “S” for Superficial, “I” for Internal and “A” for Alarming.
